(19) United States
(12) Patent Application Publication
(10) Pub. No.: US 2007/0167760 A1
(43) Pub. Date: Jul. 19, 2007

(54) ULTRASOUND IMAGING SYSTEM AND METHOD FOR FORMING A 3D ULTRASOUND IMAGE OF A TARGET OBJECT

(75) Inventors: Nam Chul KIM, Daegu (KR); Sang Hyun Kim, Busan (KR); Eui Chul Kwon, Seoul (KR); Tien Dung Nguyen, Hanoi (VN)

Correspondence Address: OBLON, SPIVAK, MCCLELLAND, MAIER & NEUSTADT, P.C., 1940 DUKE STREET, ALEXANDRIA, VA, 22314, US

(73) Assignee: Medison Co., Ltd., Hongchun-gun (KR)

(21) Appl. No.: 11/565,332

(22) Filed: Nov. 30, 2006

(30) Foreign Application Priority Data
Dec. 1, 2005 (KR) 10-2005-0116319

(51) Int. Cl. A61B 8/00

(52) U.S. Cl. 600/437

(57) ABSTRACT

There is provided an ultrasound imaging system, which includes: an ultrasound diagnostic unit for providing 3D volume data of an object and its neighboring region, wherein the 3D volume data is formed with a number of frames; a pre-processing unit for selecting a predetermined number of key frames from the frames; a segmentation unit for segmenting each of the key frames into object regions and non-object regions; a texture-based region classifying and merging unit for classifying the object and the non-object regions into object and non-object sub-regions based on the texture thereof, and removing the non-object sub-regions and merging the object sub-regions; a surface determining unit for extracting contours of the object in the key frames and determining a 3D surface of the object by connecting the contours; and a rendering unit for forming masked volume with the 3D surface and forming a 3D ultrasound image of the object by rendering the masked volume.

The present application claims priority from Korean Patent Application 10-2005-116319 filed on Dec. 1, 2005, the entire subject matter of which is incorporated herein by reference.

### BACKGROUND

1. Field

The present invention generally relates to ultrasound imaging systems, and more particularly to an ultrasound imaging system and method for forming a 3D ultrasound image of a target object.

2. Background

An ultrasound diagnostic system has become an important and popular diagnostic tool due to its wide range of applications. Specifically, due to its non-invasive and non-destructive nature, the ultrasound diagnostic system has been extensively used in the medical profession. Modern high-performance ultrasound diagnostic systems and techniques are commonly used to produce two or three-dimensional (2D or 3D) diagnostic images of a target object. The ultrasound diagnostic system generally uses a wide bandwidth transducer to transmit and receive ultrasound signals. The ultrasound diagnostic system forms ultrasound images of the internal structures of the target object by electrically exciting the transducer to generate ultrasound pulses that travel into the target object. The ultrasound pulses produce ultrasound echoes since they are reflected from a discontinuous surface of acoustic impedance of the internal structure, which appears as discontinuities to the propagating ultrasound pulses. The various ultrasound echoes return to the transducer and are converted into electrical signals, which are amplified and processed to produce ultrasound data for an image of the internal structure.

Recently, a 3D ultrasound imaging system has been developed to significantly influence obstetrical diagnosis. Such a system allowed improved observations of complex anatomical structures, surface scan analyses of defects, volumetric measurements of organs and 3D examinations of skeletons. Further, the 3D ultrasound imaging system can generate transparent views, which depict the sculpture-like reconstruction of fetal surface structures and transparent images of fetal inner anatomy.

A 3D ultrasound image of a fetus contains a significant amount of the abdominal wall and floating substances in the amniotic fluid, as well as the fetus itself. Accordingly, a fetus region must be distinguished from the 3D ultrasound image in order to obtain an uncovered view of the fetus and to reduce the amount of data. Generally, a method for distinguishing the fetus region from its surrounding region in the 3D ultrasound image includes region-of-interest (ROI) selection, surface extraction and region segmentation to remove the undesired data.

The 3D ultrasound imaging system determines the regions of interest (ROIs) enclosing an object in frames (i.e., 2D slices for forming volume data) and combines such ROIs to form a volume of interest (VOI). When manually selecting the ROI, a user selects the ROI by using editing tools to edit or compute a geometrical shape. On the other hand, an automatic selection of the ROI can be performed when priori properties of the object are known such that a statistical model of the object can be estimated. As for the surface extraction of the fetus, the fetus region can be distinguished from each frame, and the contours of the fetus are extracted and combined.

Further, the ultrasound image of the fetus changes at various stages of pregnancy. That is because the level of the amniotic fluid, the shape of the fetus and the position of the fetus in a womb vary according to the gestation stages. The automatic selection of the ROI, which requires relatively low accuracy, can be performed based on information regarding the level of the amniotic fluid and the shape of the fetus depending on the gestation stages. However, the automatic extraction of the contours, which requires relatively high accuracy, can be more complex and thus may be a challenge.

### BRIEF DESCRIPTION OF THE DRAWINGS

Arrangements and embodiments may be described in detail with reference to the following drawings in which like reference numerals refer to like elements and wherein:

FIG. 1 is a block diagram illustrating an ultrasound imaging system constructed in accordance with one embodiment of the present invention;

FIG. 2A depicts equally spaced frames forming volume data;

FIG. 2B illustrates five key frames selected from the frames shown in FIG. 2A;

FIG. 3A shows an original key frame;

FIG. 3B shows ROI determined in the key frame shown in FIG. 3A;

FIG. 3C shows an edge map obtained by performing a LoG operator on the ROI shown in FIG. 3B;

FIG. 3D shows a result of coarse segmentation;

FIG. 3E shows fine-segmented sub-regions, the pixel values of which are approximated by a mean value of intensity,

FIGS. 4A and 4B show the results of BDIP and BVLC of the fine-segmented sub-regions, respectively;

FIG. 5 shows the result obtained by merging fetus regions determined after SVM classification;

FIG. 6 shows an extracted contour of a fetus in a key frame;

FIGS. 7A to 7D show contours extracted from different key frames;

FIGS. 8A, 9A, 10A, 11A and 12A show 2D key frames obtained from original volume data;

FIGS. 8B, 9B, 10B, 11B and 12B show masked volumes formed from the 2D key frames;

FIG. 13 shows a ROC curve of SVM trained by using feature vectors in accordance with the present invention in comparison with ROC curves of SVM trained by exclusively using BDIP and BVLC;

FIGS. 14A, 15A, 16A and 17A are 3D ultrasound photographs of the fetus formed by using a conventional method; and

FIGS. 14B, 15B, 16B and 17B are 3D ultrasound photographs of the fetus formed by using the method of the present invention.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

In an embodiment of the present invention, a fetal contour is extracted from 3D volume data without user's operations by using SVM-based texture classification. The fetus floats in a non-fetus region such as amniotic fluid, which is darker than the fetus. Thus, the surface of the fetus can be extracted by removing the dark regions corresponding to the non-fetus region.

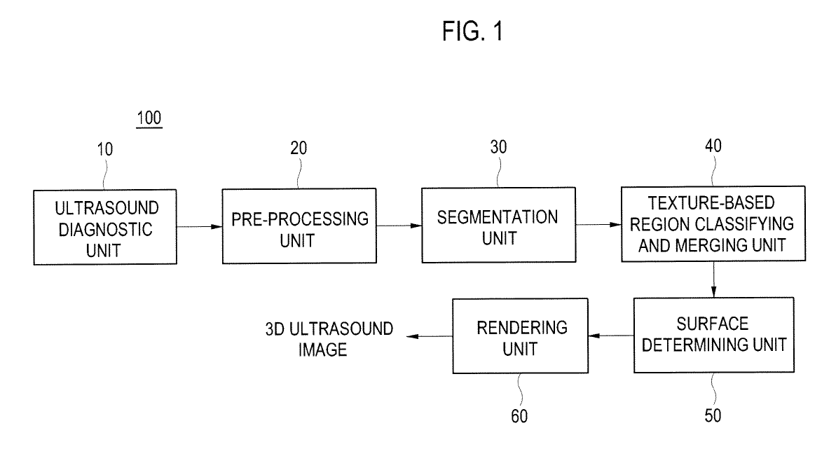

Hereinafter, one embodiment of the present invention will be described with reference to the accompanying drawings. FIG. 1 is a block diagram illustrating an ultrasound imaging system constructed in accordance with one embodiment of the present invention.

As shown in FIG. 1, an ultrasound imaging system 100 constructed in accordance with one embodiment of the present invention includes an ultrasound diagnostic unit 10, a pre-processing unit 20, a segmentation unit 30, a texture-based region classifying and merging unit 40, a surface determining unit 50 and a rendering unit 60.

The ultrasound diagnostic unit 10 provides volume data of a target object and its neighboring region. The ultrasound diagnostic unit 10 transmits ultrasound signals to the target object and forms volume data based on receive signals reflected from the target object. The volume data is formed with a number of 2D slices, i.e., frames. In an embodiment of the present invention, the target object is a fetus and the neighboring region of the fetus is the abdominal wall of a mother's body, amniotic fluid, floating substances in the amniotic fluid and the like.

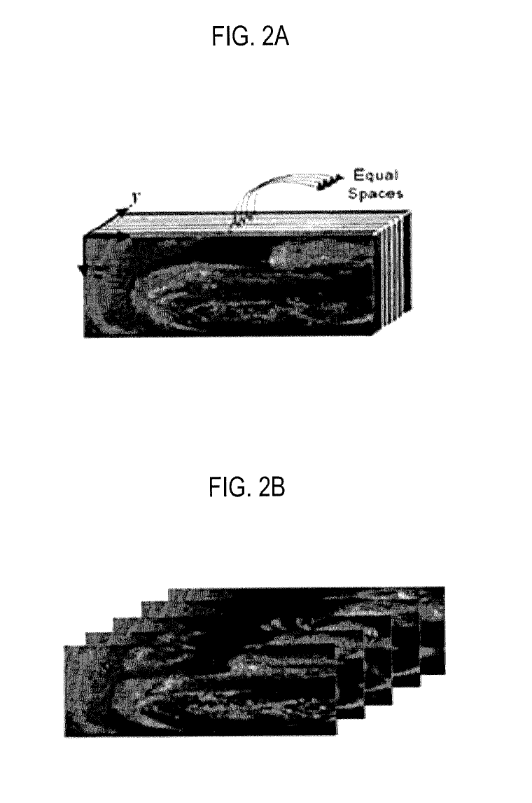

The preprocessing unit 20 selects a predetermined number of key frames from the aforementioned frames. The frames mainly including the information related to the fetus are located at a central portion of the volume data. Further, the frames rarely including such information are located at an edge portion of the volume data. Accordingly, the pre-processing unit 20 selects several frames around a central frame of the frames as key frames. Specifically, two frames located on the left and right of the central frame are selected as boundary frames such that a difference between the average intensity value of the central frame and that of the boundary frame is equal to or greater than a threshold. All frames between the boundary frames including the central frame are selected as the key frames. FIG. 2A shows the frames, which are spaced equally, forming the volume data, whereas FIG. 2B shows five key frames selected from the frames.

The segmentation unit 30 segments each of the key frames into an object region and a non-object region, namely, fetus region and non-fetus region. The segmentation unit 30 determines a region of interest (ROI) in the central frame for accurately distinguishing a fetus region from a non-fetus region. In such a case, the ROI is determined such that a left boundary of the ROI is located at an amniotic fluid region between the fetus and an abdomen of the mother's body. The ROI includes the fetus region and a portion of the non-fetus region, which includes a portion of the amniotic fluid region. The segmentation unit 30 applies the ROI to each of the key frames and performs a Laplacian-of-Gaussian (LoG) operation on the ROI in each key frame, thereby detecting a boundary between the fetus region and the non-fetus region in each ROI. The segmentation unit 30 performs coarse segmentation to remove an amniotic fluid region in the ROI of each key frame on which the LoG operation is performed. The segmentation unit 30 further performs fine segmentation to segment coarse-segmented regions into homogeneous sub-regions.

Generally, in an ultrasound image of the fetus, regions corresponding to the fetus and the abdomen of the mother's body are highly bright regions, which strongly reflect ultrasound pulses. On the other hand, the amniotic fluid region surrounding the fetus is a dark region, which absorbs the ultrasound pulses. Since there is a large difference in intensity between the fetus region and the non-fetus region, the fetus region can be found by detecting a bright region having a clear boundary. The fetus region having the clear boundary is distinguished from the non-fetus region by performing the LoG operation.

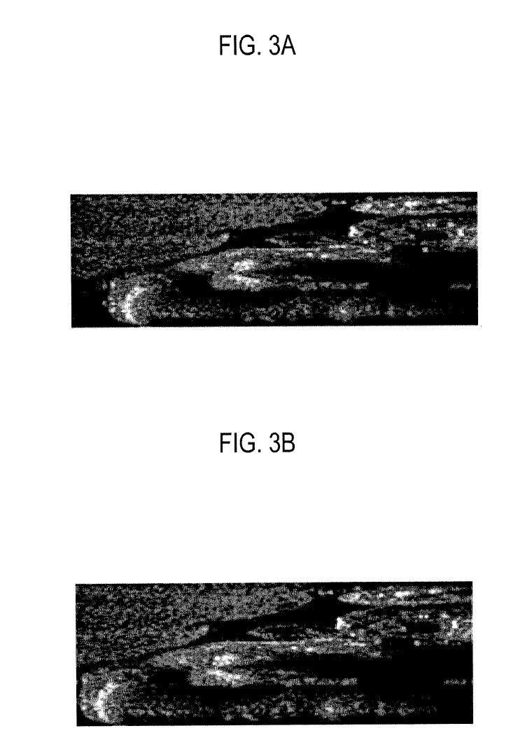

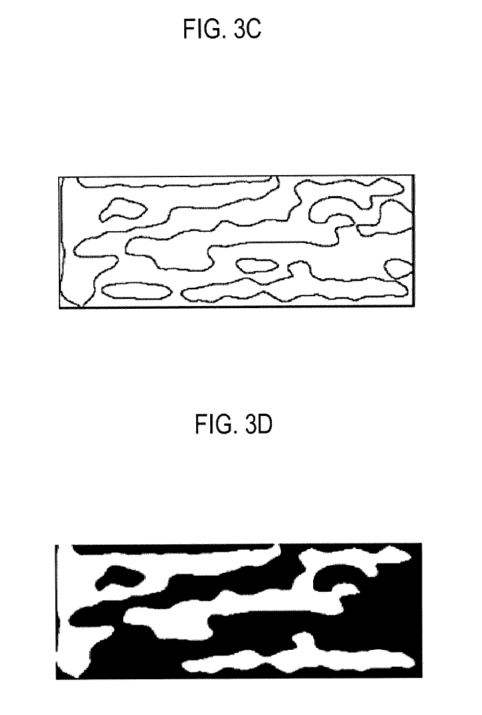

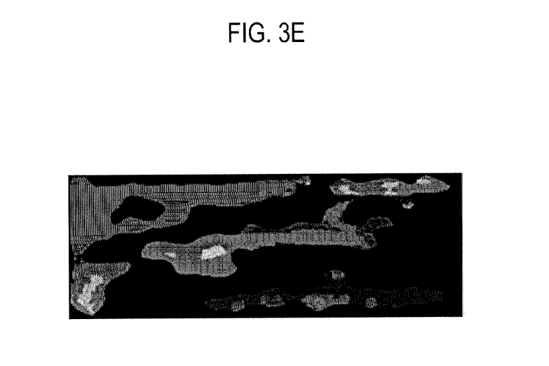

The coarse segmentation is performed to remove a dark region corresponding to the amniotic fluid region in the ROI of each key frame, which includes the fetus region and the non-fetus region distinguished from each other by the LoG operation. In this embodiment, zero-crossing detection is performed as the coarse segmentation to remove the amniotic fluid region (i.e., the dark region) from the key frame. Each key frame is segmented into the non-fetus region (abdomen of the mother's body and floating substances), the fetus region and a mixing region of the fetus region and the non-fetus region. FIG. 3A shows an original key frame and FIG. 3B shows the ROI determined by the left boundary in the key frame shown in FIG. 3A. Further, FIG. 3C shows an edge map obtained by performing the LoG operator on the ROI shown in FIG. 3B, whereas FIG. 3D shows the result of the coarse segmentation.

Fine segmentation is performed to segment the coarse-segmented regions into homogeneous sub-regions. In the ultrasound images, if the abdomen or amniotic fluid overlaps the fetus region, then an unclear edge and speckle may occur in the coarse-segmented regions. The unclear edge and speckle can be removed by using centroid linkage region growing (CLRG), wherein the coarse-segmented regions are finely segmented into homogeneous sub-regions. Accordingly, the fine segmentation leads to more accurate extraction of the fetus region.

In CLRG for segmenting the coarse-segmented regions into homogeneous sub-regions, it is assumed that homogeneity between two regions is measured by a distance between the regions. The distance from a target pixel to its neighboring region is computed by raster scanning. If the distance is short enough, then the target pixel is merged into the neighboring region. Further, if the region merged with a target pixel is close enough to another neighboring region, then the two regions are merged. In this embodiment, the CLRG employs an incremental distortion to measure the distance. In the merging process, the incremental distortion is generated, which is defined by Equation 1.
ΔD=D(R₁∪Rⱼ)−└D(Rᵢ)+D(Rⱼ)┘ Eq. 1
In Equation 1, D(Rᵢ) is a sum of squared approximation errors. When the texture of the region is approximated by a mean value, the incremental distortion is expressed by Equation 2.

$$Δ D = \frac{{N}_{i} {N}_{j}}{{N}_{i} + {N}_{j}} {( {μ}_{i} - {μ}_{j} )}^{2} \qquad Eq . 2$$

In Equation 2, Nᵢ and Nⱼ stand for the number of pixels in i^{th} and j^{th} fine-segmented sub-regions Rᵢ and Rⱼ, whereas μᵢ and μᵢ stand for mean values of intensities in the regions Rᵢ and Rⱼ.

The threshold Tᵢ for measuring homogeneity is determined for each coarse-segmented region to prevent the boundary of each coarse-segmented region from intersecting a weak edge such as unclear edge and speckle. The threshold Tᵢ is represented by Equation 3.
Tᵢ=μᵢ−c·ρᵢ Eq. 3
In Equation 3, μᵢ and ρᵢ represent a mean value and a standard deviation of intensity in the fine-segmented sub-region Rᵢ, while c represents a constant. FIG. 3E shows the fine-segmented sub-regions, the pixel values of which are approximated by the mean value of intensity.

The texture-based region classifying and merging unit 40 classifies the fine-segmented sub-regions into fetus sub-regions and non-fetus sub-regions based on the texture. It then forms a fetus image by removing the non-fetus sub-regions and merging the fetus sub-regions in each key frame. When the fetus and abdomen regions are merged, there occurs a problem in classifying the sub-regions since the fetus region and the abdomen region have a similar mean intensity. However, since fetus bones and tissues reflect ultrasound pulses at different reflection levels, the fetus and abdomen regions have different intensity variations and texture smoothness. By using block difference inverse probabilities (BDIP) and block variation of local correlation coefficients (BVLC), which are known to be efficient in image retrieval, the sub-regions may be classified into the fetus sub-region and abdomen sub-region (non-fetus sub-region). To increase the accuracy, the texture of the sub-regions can be measured by using multi-window BDIP and BVLC moments.

BDIP and BVLC will now be described below.

BDIP measures the intensity variation in a block in a frame on which the fine segmentation is performed. BDIP is defined as the ratio of a sum of values, which are obtained by subtracting the pixel values in the block from the maximum pixel value in the block to the maximum pixel value in the block Generally, BDIP of a block having the size W×W can be defined by Equation 4.

$${β}^{k} ( l ) = \frac{\frac{1}{{W}^{2}} {\sum }_{( i , j ) ∈ {B}_{l}^{k}} [ {max}_{( i , j ) ∈ {B}_{l}^{k}} I ( i , j ) - I ( i , j ) ]}{{max}_{( i , j ) ∈ {B}_{l}^{k}} I ( i , j )} \qquad Eq . 4$$

In Equation 4, I(i, j) denotes intensity of a pixel (i, j) in the block and l=(u, v), a location of the block in the image. k is defined as a maximum distance between two pixels of a pixel pair in the block and is equivalent to W−1. The BDIP increases in proportion to the intensity variation in the block

BVLC is defined as a variation of local correlation coefficients (LCCs) in a block having the size W×W according to four directions (−90°, 0°, −45° and 45°). LCC in each direction can be represented by Equation 5.

$${ρ}^{k} ( l ) = \frac{\frac{1}{{W}^{2}} {\sum }_{( i , j ) ∈ {B}_{l}^{k}} I ( i , j ) I ( i + {Δ}_{i} ( k ) , j + {Δ}_{j} ( k ) ) - {μ}_{l} {μ}_{l + Δ ( k )}}{{σ}_{l} {σ}_{l + Δ ( k )}} \qquad Eq . 5$$

In Equation 5, l=(u, v) denotes the location of the block in the image, whereas μₗ and ρₗ represent the local mean value and the local standard deviation of intensity in the block, respectively. Δ(k)=(Δᵢ(k), Δⱼ(k)) represents the position variance of the block moving in four directions. Accordingly, μ_{l+Δ(k)} and ρ_{l+Δ(k)} represent a mean value and a standard deviation of the block that is moved by Δ(k), respectively. Thus, a value of BVLC can be expressed by Equation 6.

$${γ}^{k} ( l ) = {max}_{Δ ( k ) ∈ {O}_{4}} [ {ρ}^{k} ( l ) ] - {min}_{Δ ( k ) ∈ {O}_{4}} [ {ρ}^{k} ( l ) ] , \\ {O}_{4} = \{ ( 0 , k ) , ( k , 0 ) , ( k , k ) , ( k , - k ) \} \qquad Eq . 6$$

From Equation 6, it can be seen that the larger the roughness in the block the higher the value of BVLC.

In order to better utilize the texture features of the fetus and non-fetus regions, BDIP and BVLC moments of various window sizes are calculated and combined to produce multi-window BDIP (MW-BDIP) and multi-BVLC (MW-BVLC) moments. It is known that the MW-BDIP and MW-BVLC moments have efficient texture features in image retrieval. In this embodiment, the sub-regions are classified into the fetus and non-fetus regions by using SVM. That is, the feature vectors of SVM are obtained based on the MW-BDIP and MW-BVLC moments, while the intensity variation and smoothness of tissues in the fetus and abdomen regions are characterized by using the feature vectors.

Each key frame is segmented into non-overlapping blocks of various sizes. Then, the BDIP and BVLC moments are computed in each block in a similar way as in the ROI determination. The only difference between the calculation of BDIP and BVLC moments and ROI determination is that the thresholds in block classification for calculation of BDIP and BVLC moments are determined for each segmented region. The feature vector x^{k} for a window having the size (k+1)² is defined as a combination of BDIP and BVLC moments, as shown by Equation 7.
x^{k}=[μ₁^{k}(D),μ₂^{k}(D), . . . ,μₙ^{k}(D),μ₁^{k}(V),μ₂^{k}(V), . . . ,μₙ^{k}(V)], k=1,2,3 Eq. 7
In Equation 7, μᵢ^{k}(D) and μᵢ^{k}(V) denote BDIP and BVLC means for the i^{th} class for each k, respectively. The feature vectors x^{k} (k=1, 2, 3) are computed and the MW feature vectors for SVM are obtained by the combination of the feature vectors. In order to prevent a feature vector with a large variation in SVM calculation from becoming dominant, the components of the feature vector are normalized based on the variations computed from training database (DB), as shown by Equation 8.

$$x = [ \begin{matrix}\frac{ {x}^{1}}{ {σ}^{1}} & \frac{ {x}^{2}}{ {σ}^{2}} & \frac{ {x}^{3}}{ {σ}^{3}}\end{matrix} ] \qquad Eq . 8$$

In Equation 8, component-wise division is performed. Further, σ^{k} represents the standard deviations calculated from the training DB for each k.

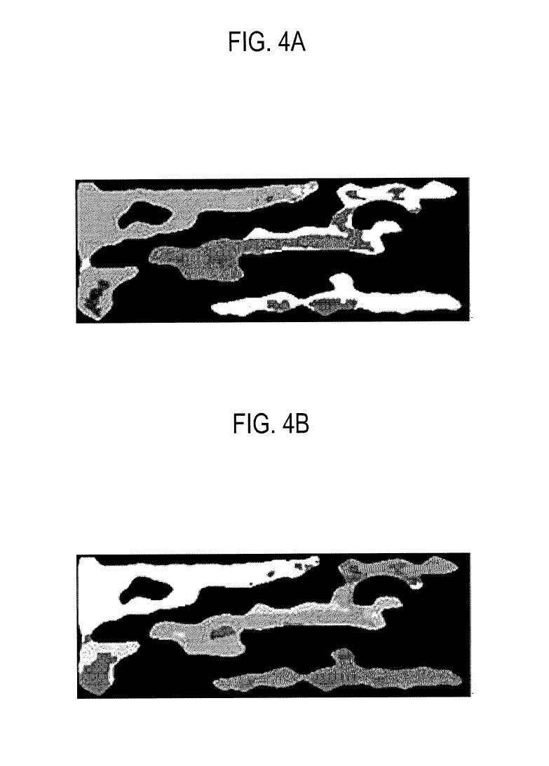

The results of BDIP and BVLC of the fine-segmented sub-regions for k=1 are shown in FIGS. 4A and 4B, respectively. The results shown in FIGS. 4A and 4B are obtained by increasing the dynamic range and equalizing the histogram to discriminate the intensity differences of the sub-regions.

When each feature vector xᵢ (xᵢεR^{N}) belongs to a class yᵢ (yᵢε{±1}), a training set of examples is referred to as {(xᵢ,yᵢ), i=1, . . . , N}. A SVM classifier is based on a hyperplane for distinguishing the classes of +1 and −1, while the distance between each class and the hyperplane is maximized. A constrained second-order optimization problem is solved by introducing Lagrange multipliers αᵢ (i=1, 2, . . . , N) so that a unique optimal hyperplane can be obtained. In such a case, solutions of the constrained second-order optimization problem can be expressed by a subset of support vectors. Linearly separable data can be obtained by analyzing the hyperplane, whereas non-linearly separable data that are generally generated in substantial separation problem can be analyzed by using an adequate non-linear operator Φ(·) for mapping input feature vectors in higher-order feature vector space. A new sample x can be classified by using a non-linear classifier, as shown by Equation 9 below.

$$\begin{matrix}f ( x ) = sgn ( {\sum }_{i = 1}^{N} {α}_{i} {y}_{i} Φ ( {x}_{l} ) Φ ( x ) + b ) \\ = sgn ( {\sum }_{i = 1}^{N} {α}_{i} {y}_{i} K ( {x}_{i} , x ) + b )\end{matrix} \qquad Eq . 9$$

In Equation 9, K stands for a kernel function satisfying the Mercer's theorem. The kernel function generally used is polynomials, Gaussian radial basic function (RBF) or tag-sigmoid. In this embodiment, an SVM classifier adopting Gaussian RBF non-linear kernel, which is relatively efficient compared to other SVM classifiers, is employed to classify the fine-segmented sub-regions.

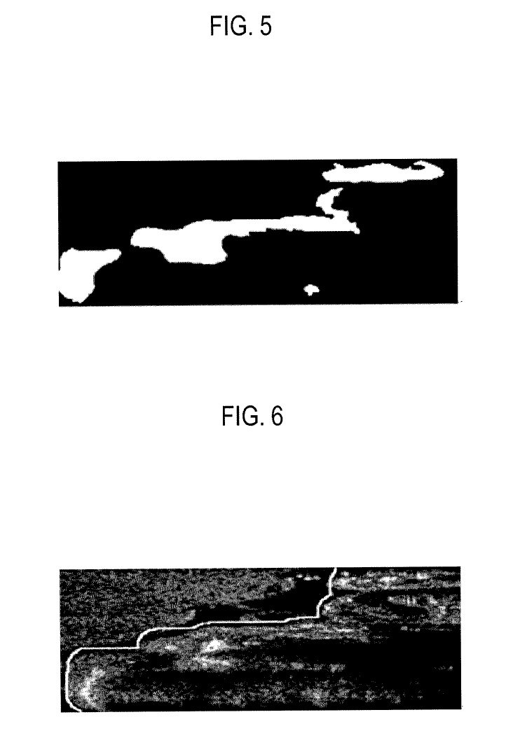

As described above, a fetus image is formed by removing the non-fetus regions from the classified sub-regions and merging the fetus regions adjacent to each other in each key frame. FIG. 5 shows the result obtained by merging the regions determined as the fetus regions after SVM classification.

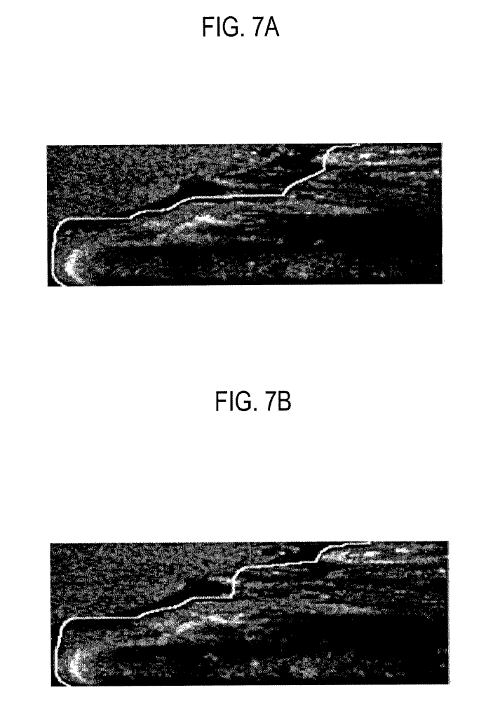

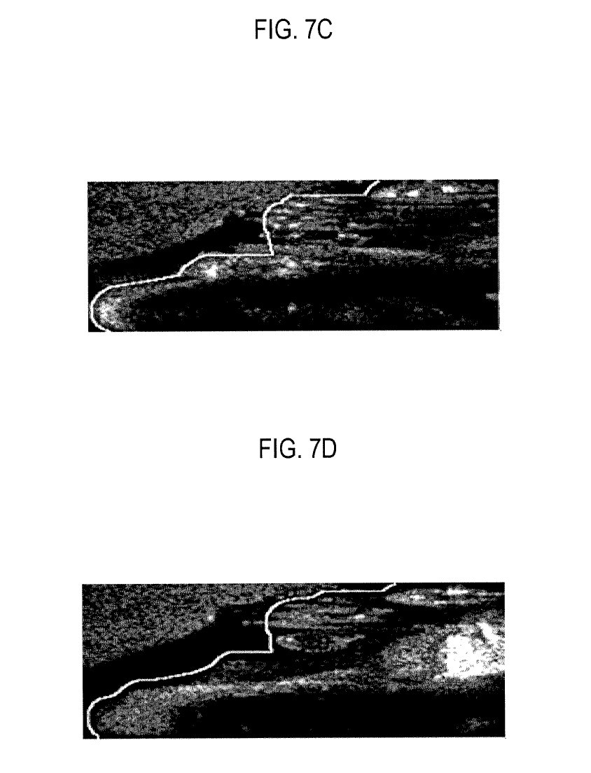

The surface determining unit 50 extracts the fetal contour in each key frame and determines a 3D surface of the fetus by connecting the extracted fetal contours. The fetal contour is extracted by finding a first pixel in each horizontal search line belonging to the surface of the fetus. FIG. 6 shows an extracted contour of the fetus in a first key frame, whereas FIGS. 7A to 7D show the contours extracted from the other key frames.

The rendering unit 60 forms a masked volume and produces a 3D image of the fetus by rendering the masked volume. The masked volume is formed only with the 3D surface in which the abdomen region of the mother's body and the floating substances are removed.

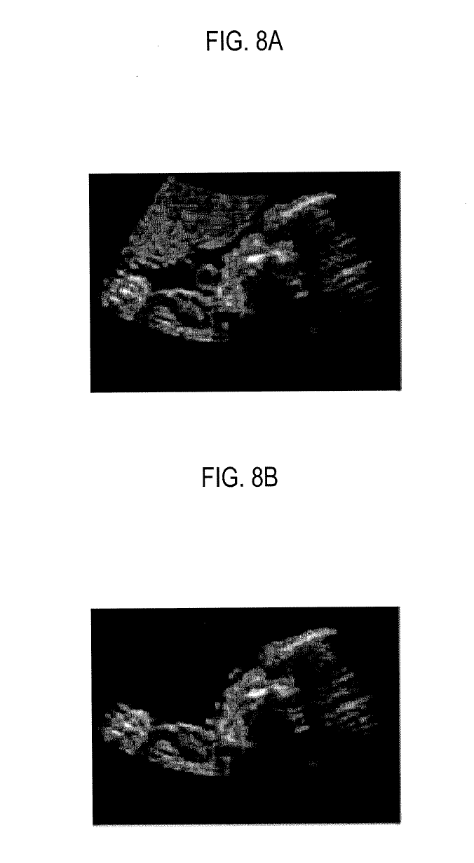

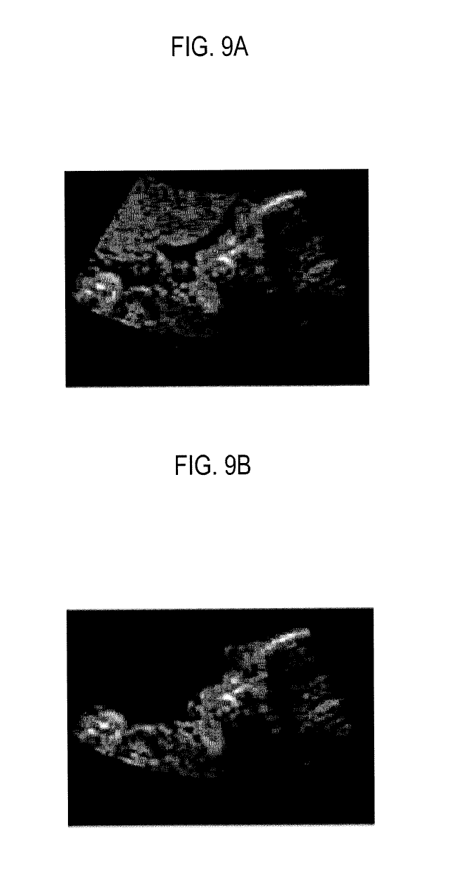

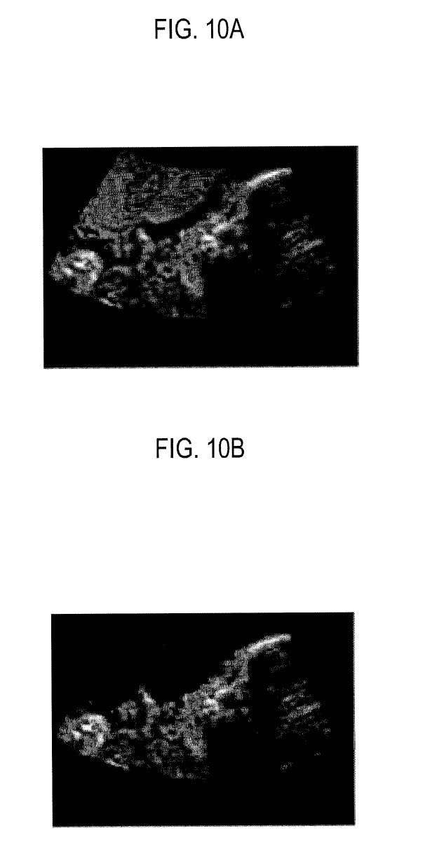

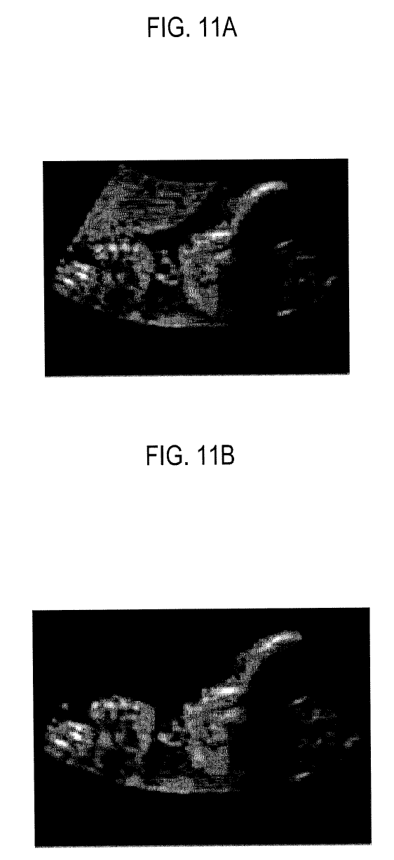

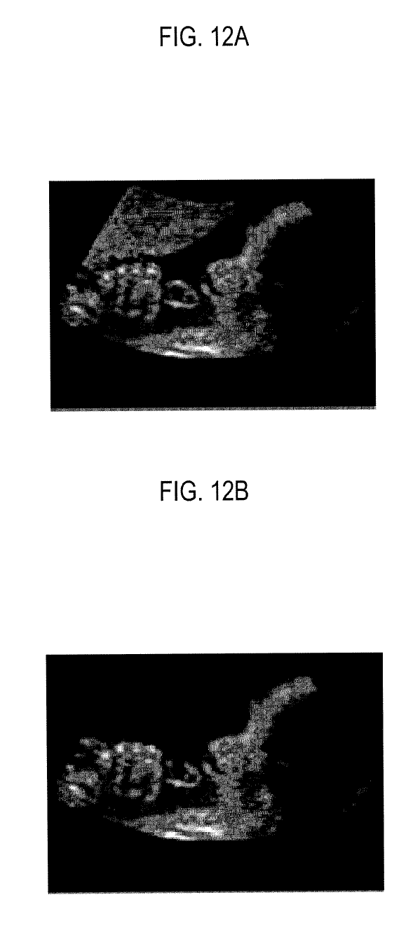

The 3D image of the fetus can be formed by using the fetus contours extracted from the key frames in consideration of spatial continuity of the fetus surface. The contours extracted from the two neighboring key frames are used for linear intrapolation of all frames inserted between the two key frames. The contours extracted from the other two frames at both ends are used to extrapolate the frames outside the two key frames. Since the intrapolated frames and the extrapolated frames contain relatively less visual information, they do not affect the quality of the image. The masked volume data are formed from the determined 3D surface while it is assumed that there is no data outside the 3D surface. FIGS. 8A, 9A, 10A, 11A and 12A show 2D key frames obtained from original volume data, whereas FIGS. 8B, 9B, 10B, 11B and 12B show the masked volumes formed from the 2D key frames.

An experimental result will now be described below.

1. Settings for Experimentation

Forty sets of raw volume data with various sizes at different gestation stages are prepared to evaluate the effects of automatic surface extraction in accordance with the present invention. A number of frames are extracted from each volume data in the sagittal direction, while 10 frames around a central frame are selected from the frames of each volume data to obtain a total of 400 frames.

In each of the 400 frames, two non-overlapping regions of size M×M are selected in the fetus region and the abdomen region of the mother's body, respectively. The sizes of the non-overlapping regions should be determined such that the non-overlapping regions do not contain a portion of the abdomen and floating substances in the frames. In this experiment, it is determined that M is 32. The selected 1600 regions are divided into two groups, each of which includes 400 fetus regions and 400 non-fetus regions.

In coarse segmentation, the LoG operator is performed with a standard deviation ρ of 9, which shows the most excellent segmentation result in the training of different ρ values. In order to remove the small regions, regions of sizes smaller than a threshold T_{Size}=100 are removed.

When calculating the BDIP and BVLC, the block size is chosen as 2×2. When selecting the SVM classifier adopting Gaussian RBF kernel, a grid search for the best parameters of width w and regularization factor C is performed by a four-fold cross-validation procedure on the training set. With the most accurate cross-validation, the SVM classifier with ρ=2 and C=15 is used for region classification. The number of classes for block classification in the calculation of BDIP and BVLC moments is determined to be 4.

2. Performance Evaluation

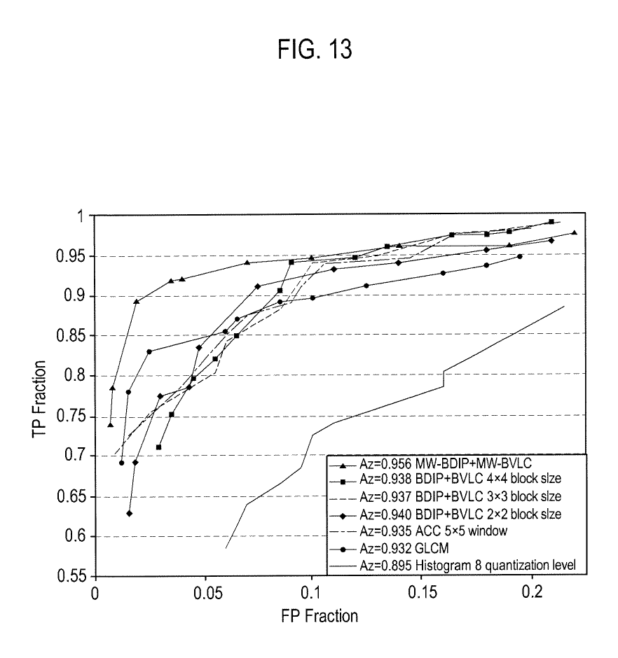

The receiver operating characteristic (ROC) analysis is used for providing a comprehensive summary of the trade-off between the sensitivity and specificity of the SVM. To investigate the contribution of BDIP and BVLC moments to the role of the proposed feature vectors, the ROC curve of the SVM trained by using the feature vectors is compared with the ROC curves of the SVM trained by exclusively using BDIP and BVLC. As shown in FIG. 13, the combination of BDIP and BVLC yields the highest SVM performance (curves under the curve A_{z}=0.94).

Table 1 represents the performance of the SVM classifier evaluated for 800 testing sets containing the fetus and non-fetus regions, wherein the accuracy, sensitivity and specificity are 93.38%, 95.5% and 91.25%, respectively. Further, when the SVM classifier is applied to 400 key frames, the number of correctly extracted contours is visually observed as 368/400 (about 92%).

TABLE 1

| Test set: 800 | Accuracy | Sensitivity | Specificity |
|---|---|---|---|
| SVM | 747/800 (93.38%) | 382/400 (95.5%) | 365/400 (91.25%) |
| performance | | | |

3. Visualization

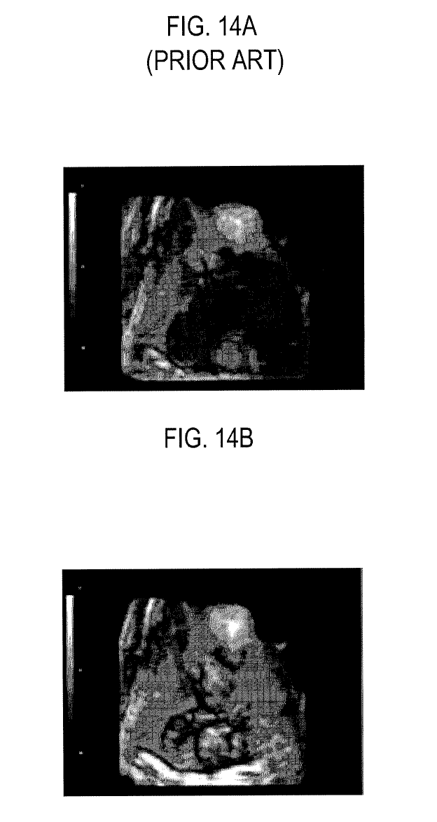

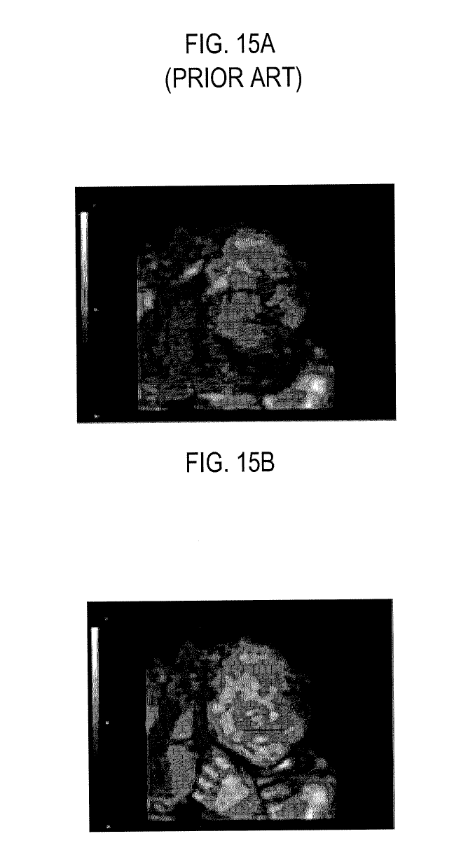

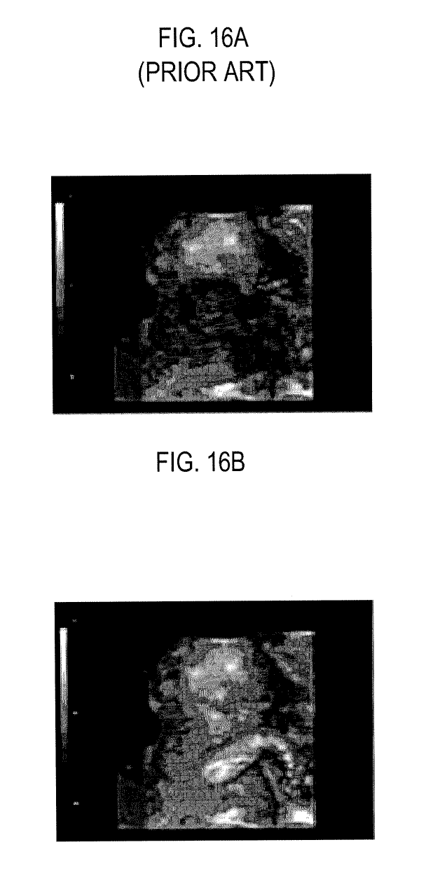

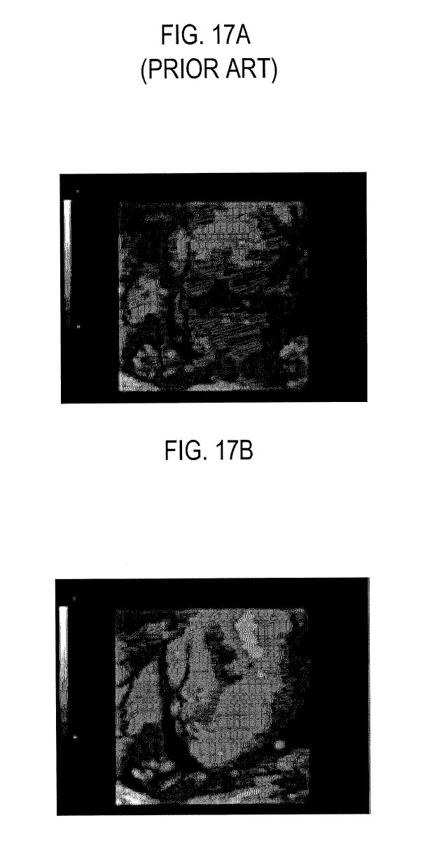

Visualization is based on the ray-casting method to form the 3D image by directly processing the VOI without visual expression. Since the data defined by the masked volume is only rendered, the system can display the ultrasound images in real time. FIGS. 14A, 15A, 16A and 17A are 3D ultrasound photographs of the fetus formed by using the conventional method, whereas FIGS. 14B, 15B, 16B and 17B are 3D ultrasound photographs of the fetus formed by using the method in accordance with the present invention.

An embodiment may be achieved in whole or in part by an ultrasound imaging system, which includes: an ultrasound diagnostic unit for providing 3D volume data of an object and its neighboring region, wherein the 3D volume data is formed with a number of frames; a pre-processing unit for selecting a predetermined number of key frames from the frames; a segmentation unit for segmenting each of the key frames into object regions and non-object regions; a texture-based region classifying and merging unit for classifying the object regions and the non-object regions into object sub-regions and non-object sub-regions based on the texture thereof, the texture-based region classifying and merging unit further being configured to remove the non-object sub-regions in the key frames and merge the object sub-regions; a surface determining unit for extracting contours of the object in the key frames and determining a 3D surface of the object by connecting the contours extracted from the key frames; and a rendering unit for forming masked volume with the determined 3D surface and forming a 3D ultrasound image of the object by rendering the masked volume.

Any reference in this specification to “one embodiment,” “an embodiment,” “example embodiment,” etc., means that a particular feature, structure or characteristic described in connection with the embodiment is included in at least one embodiment of the invention. The appearances of such phrases in various places in the specification are not necessarily all referring to the same embodiment. Further, when a particular feature, structure or characteristic is described in connection with any embodiment, it is submitted that it is within the purview of one skilled in the art to affect such feature, structure or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound imaging system, comprising:
an ultrasound diagnostic unit for providing 3D volume data of an object and its neighboring region, wherein the 3D volume data is formed with a number of frames;
a pre-processing unit for selecting a predetermined number of key frames from the frames;
a segmentation unit for segmenting each of the key frames into object regions and non-object regions;
a texture-based region classifying and merging unit for classifying the object regions and the non-object regions into object sub-regions and non-object sub-regions based on the texture thereof, the texture-based region classifying and merging unit further being configured to remove the non-object sub-regions in the key frames and merge the object sub-regions;
a surface determining unit for extracting contours of the object in the key frames and determining a 3D surface of the object by connecting the contours extracted from the key frames; and
a rendering unit for forming masked volume with the determined 3D surface and forming a 3D ultrasound image of the object by rendering the masked volume.

2. The ultrasound imaging system of claim 1, wherein the object is a fetus, the object regions are fetus regions and the non-object regions are non-fetus regions including an amniotic fluid region and an abdomen region of a mother's body.

3. The ultrasound imaging system of claim 2, wherein the segmentation unit determines a region of interest (ROI) in one of the key frames and performs the ROI on each of the key frames, the segmentation unit being configured to segment the ROI in each of the key frames into the fetus regions and the non-fetus regions by performing a Laplacian-of-Gaussian (LoG) operation and perform coarse segmentation on the ROI that underwent the LoG operation to form coarse-segmented regions and to remove the amniotic fluid region in the ROI, the segmentation unit further being configured to segment the coarse-segmented regions into homogeneous sub-regions by performing fine segmentation.

4. The ultrasound imaging system of claim 3, wherein the texture-based region classifying and merging unit segments each of the key frames into non-overlapping blocks of various sizes and computes block difference inverse probabilities (BDIP) and block variation of local correlation coefficients (BVLC) moments in each block to measure texture of the sub-regions obtained by the fine segmentation, the texture-based region classifying and merging unit further being configured to classify the sub-regions into fetus sub-regions and non-fetus sub-regions by using a support vector machine (SVM).

5. A method for forming an ultrasound image, comprising:
a) providing 3D volume data of an object and its neighboring region, wherein the 3D volume data is formed with a number of frames;
b) selecting a predetermined number of key frames from the frames;
c) segmenting each of the key frames into object regions and non-object regions;
d) classifying the object regions and the non-object regions into object sub-regions and non-object sub-regions based on texture thereof, and removing the non-object sub-region in each of the key frames and merging the object sub-regions;
e) extracting contours of the object in the key frames and determining a 3D surface of the object by connecting the contours extracted from the key frames; and
f) forming masked volume with the determined 3D surface and forming a 3D ultrasound image of the object by rendering the masked volume.

6. The method of claim 5, wherein the object is a fetus, the object regions are fetus regions and the non-object regions are non-fetus regions including an amniotic fluid region and an abdomen region of a mother's body.

7. The method of claim 6, wherein the step c) includes:
determining a region of interest (ROI) in one of the key frames;
performing the ROI on each of the key frames;
segmenting the ROI in each of the key frames into the fetus regions and the non-fetus regions by performing a LoG operation;
performing coarse segmentation on the ROI that underwent the Log operation to form coarse-segmented regions and to remove the amniotic fluid region in the ROI; and
segmenting the coarse-segmented regions into homogeneous sub-regions by performing fine segmentation.

8. The method of claim 7, wherein the step d) includes:
segmenting each of the key frames into non-overlapping blocks of various sizes;
computing block difference inverse probabilities (BDIP) and block variation of local correlation coefficients (BVLC) moments in each block to measure texture of the sub-regions obtained by the fine segmentation; and
classifying the sub-regions into fetus sub-regions and non-fetus sub-regions by using a SVM.
